(19) United States
(12) Patent Application Publication
(10) Pub. No.: US 2024/0049869 A1
(43) Pub. Date: Feb. 15, 2024

(54) High Throughput Hair Analysis for Personalized Hair Product

(71) Applicant: STRANDS HAIR CARE, Los Angeles, CA (US)

(72) Inventors: Eric DELAPENHA, Los Angeles, CA (US); Christopher DONAYRE, Hawthorne, CA (US)

(21) Appl. No.: 18/267,759

(22) PCT Filed: Dec. 23, 2021
(86) PCT No.: PCT/US2021/065125

§ 371 Date: Jun. 15, 2023

Related U.S. Application Data
(60) Provisional application No. 63/129,994, filed on Dec. 23, 2020.
(60) Provisional application No. 63/163,199, filed on Mar. 19, 2021.

(51) Int. Cl. A45D 44/00

(52) U.S. Cl. CPC A45D 44/00; A45D 2044/007

(57) ABSTRACT

A custom hair treatment provider analyzes a hair sample. The custom hair treatment provider receives the hair sample and user data from a user. The custom hair treatment provider receives a hair sample and user data from a user. The custom hair treatment provider analyzes the hair sample to produce hair analysis data and uses the hair analysis data and user data to determine, for root cause parameters, respective root cause parameter values. The custom hair treatment provider determines a particular base formula based on the root cause parameters, and generates a final formula for a custom hair treatment product based on the particular base formula.

### CROSS-REFERENCE TO RELATED APPLICATIONS

This application claims the benefit of U.S. Provisional Application No. 63/129,994, filed Dec. 23, 2020, and U.S. Provisional Application No. 63/163,199, filed Mar. 19, 2021, which are each incorporated by reference.

### FIELD OF ART

The description generally relates to analysis of hair, and in particular to analyzing a hair sample to generate a formula for a custom hair treatment product.

### BACKGROUND

The traditional process of purchasing hair products assumes that customers know their true hair condition, texture, and scalp condition. Such assumptions leave most customers confused about what products they actually need to help them achieve their hair goals. Existing products vary in provided benefits for specific hair types, damage levels, environmental factors, and lifestyle factors. The task of selecting the right product requires the customer to spend time and resources trying out different products in search of the right hair treatment products for the customer.

In general, mass-market haircare products are typically designed to be single formulas produced at the largest possible scale and sold at a high volume of customers. The problem with this approach is not all consumers have the same hair issues or needs. The market is slightly diversified by specific hair groups such as “textured hair” and “color treated hair.” Even within these kinds of segmentations, however, there still remains a need for further understanding of one's true hair condition to properly select a product.

A drawback to breaking down market segmentation even further is the ability to scale such products. Larger batch sizes equate to better economies of scale. Hence, most major organizations lean into creating single formula products and mass production.

In addition to the production limitations for consumer-segmented haircare products, oftentimes customers are not able to accurately assess their haircare needs. Customers are often unsure how to objectively self-assess their actual hair condition, texture, and scalp condition. Typically, the products created via self-assessment surveys have a high chance of resulting in custom hair treatment products that are improper to treat the customer's hair due to incorrect self-assessment by the customer.

Throughout the last decade a number of technologies have been introduced to the market that provide quantitative hair condition data to the customer. Such instruments have been used primarily in salon settings and by a hair care professional and require the customer to visit the salon professional frequently to continue to have the most up to date information about their hair condition. Handheld at home versions of these technologies have also been introduced to the market as well, but they can be difficult to use properly, and misdiagnoses can occur from improper testing conditions. Consumer-facing devices typically aim to require minimal steps in order to streamline the process for the customer; however, this comes at the expense of accuracy from improper sample preparation.

### SUMMARY

According to one embodiment of a method for generating a custom hair treatment product for a user, a custom hair treatment provider sends, to a user, a hair test kit, the hair test kit comprising a hair sample holder capable of securing hair and a hair identity questionnaire including a user data collection question. The custom hair treatment provider receives, from the user, a hair sample and user data comprising a response to the user data collection question. The custom hair treatment provider analyzes the hair sample to produce hair analysis data. The hair analysis data comprises a measure of damage to the hair sample. The custom hair treatment provider determines, based on the hair analysis data and the user data, for each root cause parameter of a set of root cause parameters, a root cause parameter value. The custom hair treatment provider determines, based on the root cause parameter values, a particular base formula of a plurality of base formulas. The custom hair treatment provider generates, based on the determined particular base formula, a final formula for a custom hair treatment product.

In an embodiment, the custom hair treatment provider generates, based on the final formula for the custom hair treatment product, a unit of the custom hair treatment product, and sends, to the user, the unit of the custom hair treatment product.

In an embodiment, the custom hair treatment provider receives, from the user, user feedback data comprising an indicator of user satisfaction with the custom hair treatment product; and adjusts, based on the user feedback data, the final formula for the custom hair treatment product.

In an embodiment, the custom hair treatment provider generates, based on the user data, the hair analysis data, and the final formula for the custom hair treatment product, a user electronic profile. The custom hair treatment provider stores the user electronic profile. The custom hair treatment provider generates, based on the user electronic profile, a user hair report. The custom hair treatment provider sends, to the user, the user hair report.

In an embodiment, the root cause parameters in the set of root cause parameters are divided into a plurality of root cause categories. The custom hair treatment provider determines, based on a first subset of root cause parameter values corresponding to a first subset of root cause categories, the particular base formula for the custom hair treatment product, and determines, based on a second subset of root cause parameter values corresponding to a second subset of root cause categories and comprising a root cause parameter value not in the first subset of root cause parameter values, a second base formula for a second custom hair treatment product for the user.

In an embodiment, determining, based on the set of root cause parameters, the particular base formula of the plurality of base formulas, comprises the custom hair treatment provider applying the root cause parameter values of the set of root cause parameters to a decision matrix, wherein one or more root cause parameters of the set of root cause parameters are criteria of the decision matrix, and one or more base formulas of the plurality of base formulas are alternatives of the decision matrix, and the custom hair treatment provider selecting, as the particular base formula, a base formula of the one or more base formulas corresponding to a highest score in the decision matrix.

In an embodiment, the custom hair treatment provider updates, based on user feedback data, one or more weights of the decision matrix.

In an embodiment, determining, based on the set of root cause parameters, the particular base formula of the plurality of base formulas, comprises the custom hair treatment provider applying the root cause parameter values of the set of root cause parameters to a trained machine learning model, wherein the trained machine learning model generates a score for each base formula of the plurality of base formulas, and the custom hair treatment provider selecting, as the particular base formula, a base formula of the plurality of base formulas with a highest score.

In an embodiment, the trained machine learning model is trained on the plurality of base formulas and respective pluralities of training sets of root cause parameter values, each training set of root cause parameter values having a respective label indicating a level of user satisfaction.

In an embodiment, the custom hair treatment provider receives user feedback data in response to the custom hair treatment product and re-trains, using the user feedback data, the particular base formula, and the root cause parameter values, the trained machine learning model.

In an embodiment, analyzing the hair sample to produce hair analysis data comprises the custom hair treatment provider performing chemical or physical analysis upon the hair sample to produce a total protein loss parameter value for a total protein loss parameter, wherein the measure of damage to the hair sample comprises the total protein loss parameter, where a particular root cause parameter of the set of root cause parameters is based on the total protein loss parameter, and a particular root cause parameter value for the particular root cause parameter is based on the total protein loss parameter value.

In an embodiment, performing the chemical analysis upon the hair sample to produce the total protein loss parameter value comprises assessing protein loss in the hair sample using a protein assay.

In an embodiment, the protein assay comprises extracting surface cuticles from the hair sample, and assessing the protein content of the extract.

In an embodiment, performing the chemical analysis upon the hair sample comprises spectroscopic analysis of the hair sample.

In an embodiment, performing the chemical analysis upon the hair sample comprises optical microscopy on the hair sample.

In an embodiment, performing the chemical analysis upon the hair sample comprises assessing one or more physical properties of hair from the hair sample.

In an embodiment, the one or more physical properties are selected from tensile strength, hair thickness, and hair gloss.

In an embodiment, analyzing the hair sample to produce hair analysis data comprises the custom hair treatment provider performing imaging analysis upon the hair sample to produce a cuticle image parameter value for a cuticle image parameter, wherein the measure of damage to the hair sample comprises the cuticle image parameter, where a particular root cause parameter of the set of root cause parameters is based on the cuticle image parameter, and a particular root cause parameter value for the particular root cause parameter is based on the cuticle image parameter value.

In an embodiment, performing the imaging analysis upon the hair sample to produce the cuticle image parameter value comprises the custom hair treatment provider generating, based on the hair sample, a cuticle image, applying the cuticle image to a trained machine learning model, wherein the trained machine learning model generates a cuticle image score, and determining the cuticle image parameter value based on the cuticle image score.

In an embodiment, the root cause parameters in the set of root cause parameters are divided into a plurality of root cause categories, and the custom hair treatment provider determines, based on a first subset of root cause parameter values corresponding to a first subset of root cause categories, the particular base formula for the custom hair treatment product, and determines, based on a second subset of root cause parameter values corresponding to a second subset of root cause categories and comprising a root cause parameter value not in the first subset of root cause parameter values, a second base formula for the custom hair treatment product, where generating the final formula for the custom hair treatment product is further based on the second base formula.

In another embodiment, a non-transitory computer-readable storage medium stores instructions that, when executed by a computing device, executes one or more steps of one or more of the above-described methods.

In yet another embodiment, a computer system includes a processor and a non-transitory computer-readable storage medium that stores instructions for executing one or more steps of one or more of the above-described methods.

### BRIEF DESCRIPTION OF THE DRAWINGS

FIG. 1 illustrates a system environment in which the techniques described herein may be practiced, according to an embodiment.

FIG. 2 is a block diagram illustrating a custom hair treatment provider, according to an embodiment.

FIG. 3 is an index figure illustrating the proper arrangement of FIG. 3A-D, which together illustrate a data flow diagram of a technique performed in the system environment, according to an embodiment.

FIG. 3A-D illustrate a data flow diagram of a technique performed in the system environment, according to an embodiment.

FIG. 4 is a flowchart illustrating steps of a technique performed in the system environment, according to an embodiment.

FIG. 5 is a block diagram that illustrates a computer system upon which embodiments of components of the system environment depicted in FIG. 1 may be implemented, according to an embodiment.

### DETAILED DESCRIPTION

### Overview

Before describing the invention in detail, it is to be understood that the invention is not limited to specific hair types or morphologies, which may vary. It is also to be understood that the terminology used herein is for the purpose of describing particular embodiments only and is not intended to be limiting. In addition, as used in this specification and the appended claims, the singular article forms “a,” “an,” and “the” include both singular and plural referents unless the context clearly dictates otherwise. Thus, for example, reference to “a hair sample” includes a single hair sample as well as a plurality of hair samples, reference to “a cuticle” refers to a single cuticle as well as a collection of cuticles, and the like.

In this specification and in the claims that follow, reference will be made to a number of terms that shall be defined to have the following meanings, unless the context in which they are employed clearly indicates otherwise: The term “hair” is used in its ordinary sense and refers to any of the fine threadlike strands growing from the skin of humans, mammals, and some other animals. The term “scalp” is used herein in its ordinary sense and refers to the skin covering the head, excluding the face. The term “high-throughput” is used herein to refer to a method for scientific experimentation and/or analysis. Using techniques that is commonly associated with robots, data processing/control software, liquid handling devices, and sensitive detectors, etc., high-throughput screening allows for a firm, company, or other practitioner of the invention to quickly conduct numerous of chemical, structural, and/or textural tests, often in parallel. The results of such experiments and/or analyses may provide starting points for hair care product design and/or productions for a plurality of customers with differentiated individualized needs.

In general, the technology described herein is a high throughput hair analysis methodology, that yields accurate global data from a customer hair sample and provide this data to the customer, a report being one example. Customers simply collect a hair sample from their comb, recent haircut, or in shower shedding, and ship for analysis. The hair is analyzed for cuticle protein loss which provides more dynamic information about the hair quality and integrity. Measuring cuticle protein loss is a standard methodology used in research labs and is directly correlated to the perceived hair properties such as shine, frizz, breakage, and split ends. The benefit of this approach is that it measures that it accounts for cuticle weakening. By using an extraction step, a hair sample's surface layer cuticles are liberated and then the quantity of loss from the sample is correlated.

When hair is damaged, typically there are different degrees of cuticle weakening, meaning two different fibers with different degrees of damage could appear to have the same cuticle condition using standard visual techniques. However, when analyzed for cuticle loss it becomes apparent that cuticle integrity is different relative to the types of conditions the hair sample has been exposed to prior. They may, for example, exhibit different tensile strengths reflected in different stress-stain curves. Another example of the impact of damage exposure is on the chemical identity of the hair. This can also be examined using chemical analysis techniques such as spectroscopic methods.

Unlike more handheld or in person devices, the technology described herein is not limited by the location of the customer nor requires the customer to be present when analysis is conducted. Moreover, the inventive technique described does not require the hair sample to be neatly preserved or aligned in order to capture proper results unlike some of the handheld devices. Humans shed between 50-200 strands of hair daily, so the sample process is reduced to simple collection from a brush/comb, a shower drain, and/or freshly cut hair.

Human hair fibers generally are composed of three main layers, the cuticle, the cortex, and the medulla. The cuticle layer is the outer most layer on the fiber and typically it is the layer that is exposed to most external damage. The structure of the cuticle layer is much like shingles on a roof with overlapping plates tightly locked into one another. When the cuticle suffers damage such as lift, chipping, pitting, and even total loss, this results in consumer perceived issues such as loss of shine, frizz, breakage, and split ends. Typically, the cuticle layer will become more labile with it is exposed to damaging conditions, and although visually it might appear that the cuticle is intact, the inventive analysis helps isolate samples with true high cuticle integrity from sample with lower cuticle integrity.

### Example Sources of Hair Damage

There are many sources in which hair can become damaged or compromised. Regardless of the source, the implications on the hair results in great sensorial issues for consumers today such as frizz, loss of shine, reduction in moisture/dry feeling hair, breakage, and split ends to name a few. There are a host of external sources that could be damaging to the hair as well as direct sources of damage. Although consumers typically have an understanding the direct treatment that they have exposed their hair to, many times they don't understand the full picture. Below is a table of examples of some of the various external (environmental or others) and direct damaging exposures, according to an embodiment. All these different potential sources of damage could compromise the hair integrity which would result in changes in the biochemical and bulk physical properties of the hair.

| Source | Treatment | Environmental | Other |
|---|---|---|---|
| UV Exposure | No | Yes | No |
| Humidity | No | Yes | No |
| Water Hardness | No | Yes | No |
| Particle Pollution | No | Yes | No |
| Other Pollution | No | Yes | No |
| Bleach | Yes | No | No |
| Permanente Hair Color | Yes | No | No |
| Semi-Permanente Hair Color | Yes | No | No |
| Natural Hair Color | Yes | No | No |
| (example: Henna) | | | |
| Permanent Straightening, | Yes | No | No |
| Curling, Waving | | | |
| Relaxers | Yes | No | No |
| Chemical Smoothing Treatment | Yes | No | No |
| Direct Heat Styling | Yes | No | Yes |
| In Direct Heat Styling | Yes | No | Yes |
| Shampooing/Washing | Yes | No | No |
| Chlorine | No | Yes | No |
| Salt Water | Yes | Yes | No |
| Combing/Brushing Hair | Yes | No | No |
| Friction from Contact | No | Yes | No |
| (pillowcases, towels) | | | |
| Styling | Yes | No | Yes |
| Hormonal Changes | No | No | Yes |
| Medical Causes | No | No | Yes |
| Psychological Causes | No | No | Yes |
| Ageing | No | No | Yes |

### Details of High-Throughput Methodology

Hair samples are analyzed for protein loss by a standard protein assay or a customized protein assay. Typically, hair samples are extracted to remove any labile surface cuticle. Methods used to extract the surface cuticles have been described in prior literature, however these methods have not been optimized to be utilized in a high throughput fashion. Optimized extraction shake times, precision shaking speeds, and maximum sample loading are utilized in order scale extraction process. After the surface cuticle is extracted from the hair sample, an aliquot of the supernatant is analyzed using a standard protein assay methodology, previously found in the prior art. Protein loss is quantified and directly correlated to a rating scale to provide to the customer a cuticle integrity score. This score is directly correlated to the level of hair damage the customer has experienced.

In some embodiments, the hair that has been extracted from can be tagged with some of dye or florescent compound to evaluate for direct cuticle loss from the actual hair sample. Image analysis method could be used to quantify the level of loss and directly correlated to a cuticle loss rating scale.

TABLE 1
Examples of Hair Testing Methods

| Hair Testing | Information Provided |
|---|---|
| Protein Assay | Protein Loss/Cuticle Integrity/Fragility |
| Microscopy | Surface Visualization, Surface Roughness, Surface |
| | Quality, Inner Fiber Visualization/chemical identity |
| Spectroscopy | Chemical Identity, Water Content/Type, Disulfide |
| | Bonds/Cysteic Acid content, 18-methyl eicosanoic |
| | acid (18-MEA) presence |
| Physical Properties | Tensile Properties, Viscoelastic properties, |
| | Thermo-analytical, |
| Surface Properties | Hydrophobicity, Surface Energy, Charge Density |
| Expert Testing | Stylist/Expert grading |
| Consumer Testing | Perception |

TABLE 2
Examples of Scalp Testing Methods

| Scalp Testing | Examples of Information Provided |
|---|---|
| Testing Strips | Scalp Sebum/Oil Presence, Dandruff Presence, |
| | Moisture Levels, pH |
| Microscopy | Visualization of surface, hair density, dandruff |
| Moisture Measurements | Hydration levels, Moisture |
| Surface Biology | Microbiome population, bacterial conditions, |
| | scalp sebum production, hair follicle quality |
| Expert Testing | Stylist/Expert grading |
| Consumer Testing | Perception |

Using a device to directly assess condition and prescribe specific ingredients is common amongst the skin care category because this category is more regulated due to skin being a living organ. The hair category requires a different approach to achieving a product that results in customer satisfaction. Hair products are generally designed for hair and scalp needs. Sensory aesthetics play a major role in the consumer acceptance in the product performance Therefore, in addition to proper diagnosis of direct factors such as hair condition, understanding of indirect factors such as lifestyle, regional/environmental factors, and habits is also important to understand each customer specific perception factor such as aesthetic needs.

In an embodiment, the invention utilizes direct customer data collected from a pre-survey, customer hair analysis data that is collected using quantitative methods, and provides a diagnosis of the customer hair condition. The technology is able to provide the customer with their true data and generate personalized hair product formulations and recommendations. The technology described starts with understanding customer hair concerns followed by analyzing a hair sample from the customer to identify hair condition, hair texture, and cuticle condition. Assessing these metrics quantitatively eliminates the subjectivity of self-assessment. Based on the hair testing results the technology is able to produce hair condition details for the customer and generate personalized formulations and recommendations.

A customer may notice that she/he is unable to find the right hair care products to meet their hair care needs. In such a case, the customer may purchase a test kit which is comprised of scalp testers, fragrance cards, and a sample collection bag. The customer activates the test kit online either using a computer, phone, or another online device and answers a number of questions ranging from perceived hair condition, environmental/regional factors pulled from their zip code, lifestyle questions, and behavioral factors such as specific treatments, products, and tools used with frequency. The customer is prompted to conduct a scalp sebum testing using scalp sebum test strips and select level within the questionnaire. Next the customer is able to smell the scent options from the scent cards and enter preference during activation. Finally, the customer is prompted to collect a hair sample and send back to the analysis labs. Once sample is received, the analysis labs can conduct one of the many hosts of testing methods to understand hair condition, properties, and hair texture. All data collected from analysis and activation are stored in a specific software system that is also equipped to assign ingredients and provide recommendations based on the whole customers hair profile. The customer receives a full hair analysis report, recommendations, and finished products based on hair profile results.

Then, according to the embodiment, the customer's data is stored in a cloud-based databank, and re-analyzed seasonally to adjust for regional/environmental changes. In addition, the customer is also able to update data based on changes in habits, routines, and lifestyle. The system is able to auto-update, based on these changes and adjust recommendations, product, and ingredient needs. Additional updates are made to the system when new ingredient technologies are introduced, ingredient dosage data, and ingredient interaction information is identified.

### Hair Recommendation Process:

Multiple data sets are collected from each customer, below is an example of the types of data sets collected, but are not limited to these data sets.
1. Questionnaire
   a. Demographics
   b. Hair & Scalp Concerns/Issues
   c. Lifestyle
   d. Hair & Scalp Habits
   e. Regional/Environmental Factors—can be sourced from zip code
   f. Allergies and Ingredient No List
   g. Product Sensory Attributes Preferences
2. Interactive Selection
   h. Trial samples of specific attribute and select such as scent options.
   i. Image of Hair and Scalp to answer specific question such as but not limited to hair type, hair density, and scalp condition.
3. Analysis
   j. Scalp Condition such as but not limited to sebum levels, dandruff levels, moisture levels, microbiome details, etc.
   k. Hair Condition
   l. Hair Texture
   m. Hair Type
   n. Hair Density

Once all this data is collected, it is processed through an algorithm that factors all the specific inputs and classifies the customers total hair and scalp profile based on the inputs. The system uses the specific customer data and applies it to decision matrix to properly select product and ingredient assignment as well as recommendations. In addition, the algorithm continues to cluster all customer data, feedback, and profile changes in order to continue to improve product/ingredient assignment and recommendations.

In some embodiments, the technology includes collecting data specific to the customers environmental & regional factors, specific hair concerns, current treatments used, allergies, lifestyle, diet, habits, and used when creating the personalized product formulations and recommendations.

In some embodiments, the technology is able to provide customers with refined products based on feedback that is updated in their hair profile. The feedback is additionally used to continue to refine and improve the product assignment algorithm.

In some embodiments, scalp analysis is conducted and used to product scalp condition details for the customer and generate personalized formulations and recommendations.

In some embodiments, additional scalp data is collected through direct measurement and/or scalp condition history. In some embodiments, sensorial functionality can be custom built into the finished good such as scent, texture, rinse off time, etc.

In some embodiments, cross ingredient interactions are considered and recommendation is adjusted.

In some embodiments, the customer can request for specific ingredients to be removed from products and/or recommendations.

In some embodiments, the customer is able to directly measure different regional and environmental factors around the world that may affect hair and scalp properties.

### EXAMPLES OF TECHNIQUES USED TO EXTRACT HAIR SAMPLES

### Example 1

A sample is extracted using a multi-arm wrist shaker set-up for a period of time to allow for only surface cuticle removal. When virgin hair is compared to damaged hair, the extracted protein concentrations are significantly lower than in the damaged hair. The reduced protein concentration from the virgin hair relative to the damaged sample is just one example of how labile cuticle extraction delivers a method to analyzing the total hair damage across the entire sample without the customer present.

### Example 2

A more rapid approach to extracting the labile surface cuticle layer from hair is by simply increasing the total RPM used during the extraction process. A vortex mixer is another example of extracting labile surface cuticles from hair samples at even faster speeds which would results in less required extraction times.

### Example 3

More concentrated hair sample to water used in the extraction process is another approach to reducing the time it takes to extract labile surface cuticle from the hair samples. By simply reducing the volume of water used, lower extractions times are also achieved.

Factors that Drive Extraction Efficiency

In order to optimize the throughput for the analysis process, one of the main approaches used is to play with a few of the variables in the process, for example:
Volume of liquid used in extraction
Extraction shaking speeds
Types of shaking/mixing used
Shape of the vessel used in the extraction process
Temperature during extraction
Pressure used during extraction
Amount of actual hair sample used during extraction Optimization of these parameters results in a process that is high volume, quick, high throughput, and able to commercialize readily as a consumer facing testing methodology.

In some instances, the hair samples must be pre-washed prior to testing to ensure proper test results. In order to wash the hair samples in a high throughput fashion a number of techniques can be employed.

### Example 4

Rapid washing using a cup equipped with a mesh filter cap, hair samples placed inside the cup and washed and rinsed multiple times without causing additional damage the sample. The mesh cap allows for easy draining and rinsing of the sample.

### Example 5

Samples are placed into a washing device in which a wash cycle and rinse cycle exposes multiple samples as once while remaining gentle enough not to cause additional damage to the sample.

### Example 6

Samples are suspended into a trough that cycles wash and rinse cycles in a gentle fashion in order not to damage the hair sample.

### Example 7

A customer places their hair sample in to a cleansing liquid that is compartmentalized in a tube or some sort of package that prevents the liquid from spilling. Once the sample arrives to the lab the sample is technically clean and can be analyzed immediately.

### Example 8

The hair sample that is collected is place by the customer is placed into a container that has cleaning solution. Once the sample arrives to analysis facility, the sample is simply removed from solution, rinsed, and analysis is conducted.

Thus, the invention allows for the utilization of hair analysis in a nonobvious commercially viable manner, through the use of a high-throughput system for analyzing hair and optionally scalp-related samples.

### System Environment

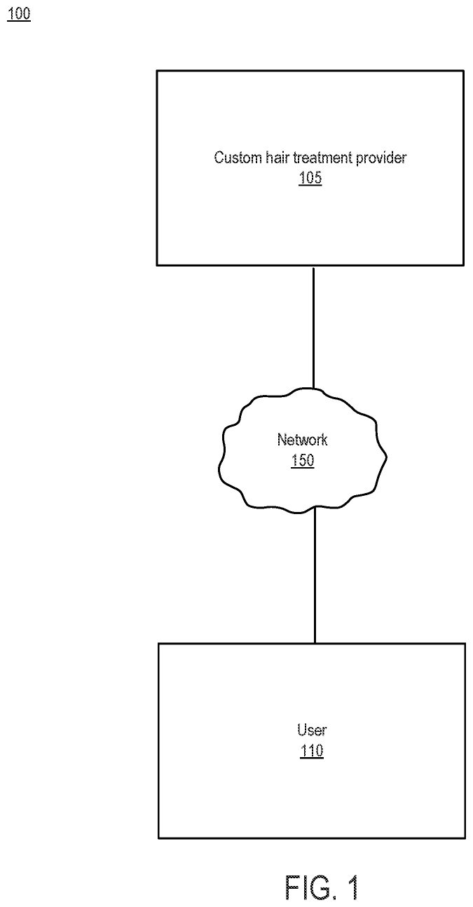

FIG. 1 illustrates a system environment 100 in which the techniques described herein may be practiced, according to an embodiment. In the embodiment shown, the system environment 100 includes a custom hair treatment provider 105 and a user 110, which may be connected via a network 150. In different embodiments, the system environment 100 and its components may include different or additional elements than those illustrated. Furthermore, the functionality may be distributed among the elements in a different manner than described. The system environment 100 comprises components that are implemented at least partially by hardware at one or more computing devices, such as one or more hardware processors executing stored program instructions stored in one or more memories for performing the functions that are described herein. In other words, some or all functions described herein are intended to indicate operations that are performed using programming in a special-purpose computer or general-purpose computer, in various embodiments. The components of FIG. 1 are now described in more detail.

The custom hair treatment provider 105 is an entity, such as a business, university, charity, non-profit organization, or other organization, that includes a computing device. The custom hair treatment provider 105 generates custom hair treatments for users based on user data and hair analysis data. The user data is data representing one or more factors that can impact hair health. The hair analysis data represents the results of analysis of a user hair sample. The custom hair treatment provider 105 sends a hair test kit to the user 110 that includes a hair sample holder and a hair identity questionnaire including a user data collection question. The custom hair treatment provider 105 receives a hair sample and user data from the user 110, e.g., a response to the hair identity questionnaire and a hair sample secured within the hair sample holder. The custom hair treatment provider 105 analyzes the hair sample to produce the hair analysis data which, together with the user data, the custom hair treatment provider 105 employs to generate a formula for a custom hair treatment product. As illustrated by the various embodiments detailed herein, myriad techniques may be employed by the custom hair treatment provider 105 as part of generating a custom hair treatment product, some or all of which may be employed together. Various techniques and embodiments described herein may be employed by the custom hair treatment provider 105, individually or together, unless the description herein explicitly indicates otherwise, and/or unless such individual or cumulative use would be impossible.

The user 110 is an entity, such as a person, and can also include a computing device, such as a personal computer (e.g., a desktop computer, laptop, smartphone, tablet, or so on). The user 110 has hair, which the user 110 collects to generate the hair sample. The user 110 sends the hair sample and user data to the custom hair treatment provider 105.

In an embodiment, user data includes both user data received from the user 110 (e.g., in response to one or more user data collection questions) and user data determined by the custom hair treatment provider 105 using one or more third parties. The user data determined by the custom hair treatment provider 105 using one or more third parties may be determined using user data received from the user 110. For example, the custom hair treatment provider 105 may generate a query using received user data and send the query to a database at a third party, and in response, the custom hair treatment provider 105 receives additional user data. As a particular example, received user data may include a geographic location, and the custom hair treatment provider 105 may query one or more third parties based on the geographic location to acquire environmental data relevant to the user 110 (e.g., environmental data representing environmental factors that can affect hair health and to which the user 110 is proximate). In an embodiment, the user 110 sends an image of the user's 110 hair to the custom hair treatment provider 105, e.g., as part of the user data.

In an embodiment, the custom hair treatment provider 105 analyzes the hair sample to produce the hair analysis data by performing one or more analyses to determine protein loss. In another embodiment, the custom hair treatment provider 105 analyzes the hair sample to produce the hair analysis data by performing spectroscopy upon the hair sample, and further employs one or more analytical techniques to determine protein loss for the hair sample in order to confirm the results of the spectroscopic analysis.

The network 150 connects the custom hair treatment provider 105 and the user 110. For example, the custom hair treatment provider 105 may send the questionnaire to the user 110 via the network 110, and receive user data from the user 110 via the network. The network 150 may be any suitable communications network for data transmission. In an embodiment such as that illustrated in FIG. 1, the network 150 uses standard communications technologies or protocols and can include the internet. In another embodiment, the entities use custom or dedicated data communications technologies. In an embodiment, the system environment 100 does not include a network 150, e.g., where the questionnaire is a paper document sent via mail to the user 110.

### Custom Hair Treatment Provider

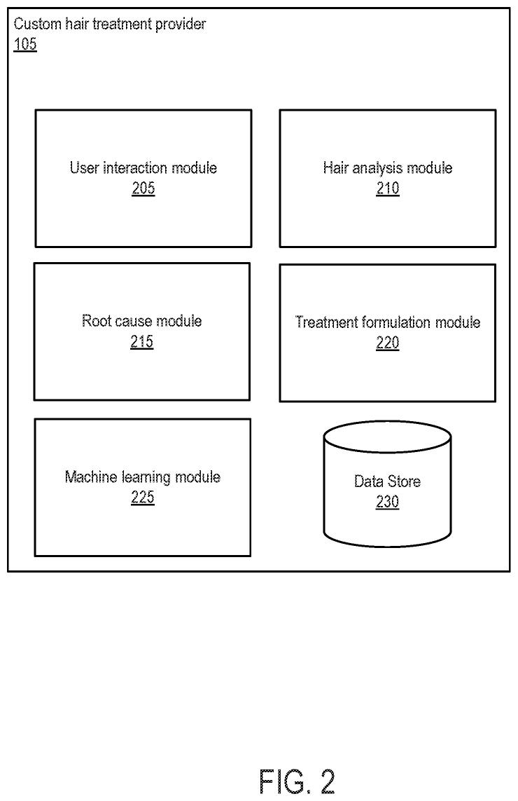

FIG. 2 is a block diagram illustrating the custom hair treatment provider 105, according to an embodiment. The custom hair treatment provider 105 includes a user interaction module 205, a hair analysis module 210, a root cause module 215, a treatment formulation module 220, a machine learning module 225, and a data store 230.

The user interaction module 205 generates user interfaces with which users 110 can interface with the custom hair treatment provider 105, e.g., to receive a questionnaire, or to send user data. The user interaction module 205 may send one or more user interfaces to the user 110 (e.g., a computing device of the user 110), such as in response to a web request sent by the user 110. The user interaction module 205 receives and/or retrieves user data and stores the user data in the data store 230. In an embodiment, the user interaction module 205 formats and/or modifies some or all user data before storage in the data store 230, e.g., to standardize the user data for use by other modules. In an embodiment, the user interaction module 205 queries third parties to acquire additional user data. In an embodiment, the user interaction module 205 generates user profiles based on user data, hair analysis data, and a final formula for the user's custom hair treatment product. The user interaction module 205 may expose some or all of the user profile to the user 110, e.g., by generating a report and sending the report to the user 110. The user interaction module 205 may also receive user feedback data from the user 110. The user interaction module 205 may interact with another component of the custom hair treatment provider 105 and/or a third party to generate a custom hair treatment product using a respective final formula and send the custom hair treatment product to the user.

The hair analysis module 210 performs one or more analytical techniques using the hair sample to generate hair analysis data. Various analytical techniques described herein involve analysis of the hair sample to determine protein loss, though in some embodiments the analysis may additionally or alternatively include imaging analysis and/or spectroscopy. Hair has a high protein content with about 300 proteins identified so far. Hair proteins (e.g., keratin, keratin-associated proteins (KAPs), and intermediate filament proteins) can be extracted from hair samples using detergent or detergent-free techniques.

Quantitation of hair protein (and thus protein loss relative to a control sample or standard) from an extracted hair sample can be achieved using any convenient techniques for protein measurement in biological samples. In some embodiments, the protein concentration in a given solution containing hair protein is determined by a colorimetric assay, such as a method described by S. S. Sandhu and C. Robbins (“A simple and sensitive technique, based on protein loss measurements, to assess surface damage to human hair.” J. Soc. Cosmet. Chem., 44, 163-175 (1993)), or by Lowry et al. (“Protein measurements with Folin phenol reagent.” J. Biol. Chem., 193, 265-275 (1951)).

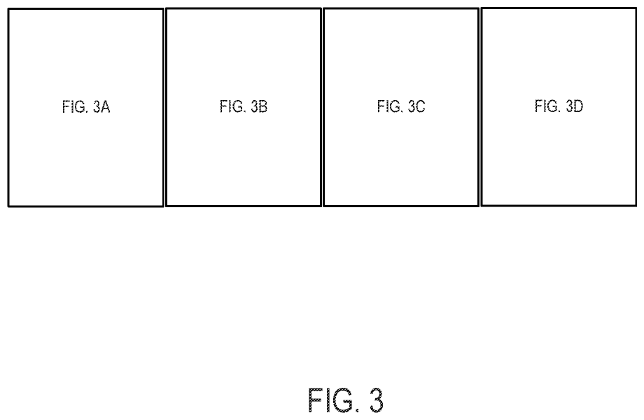

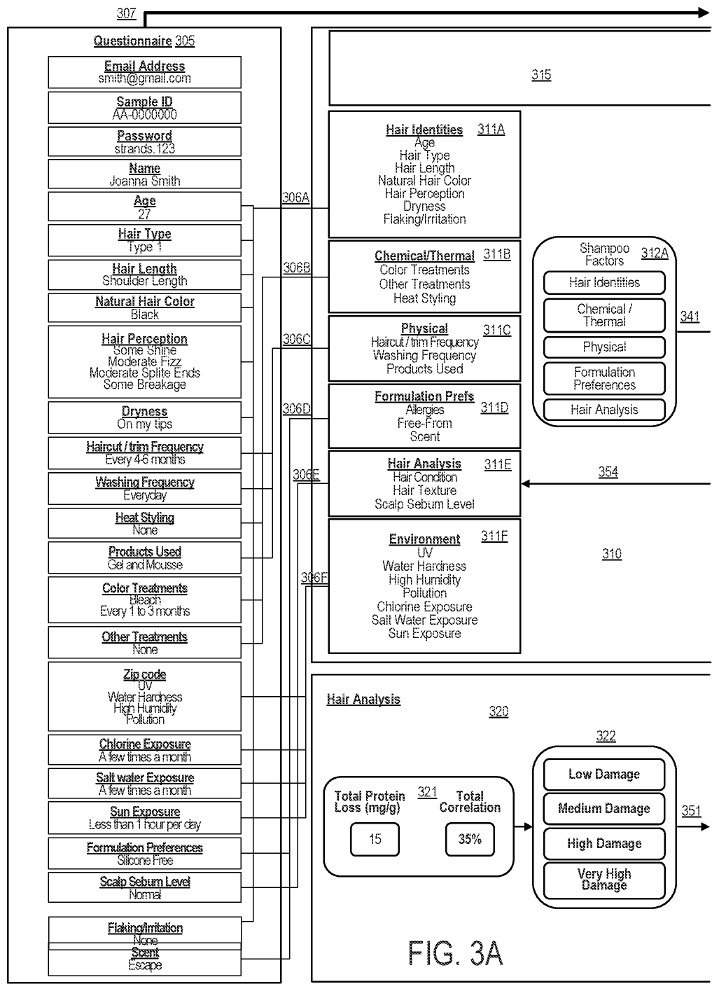

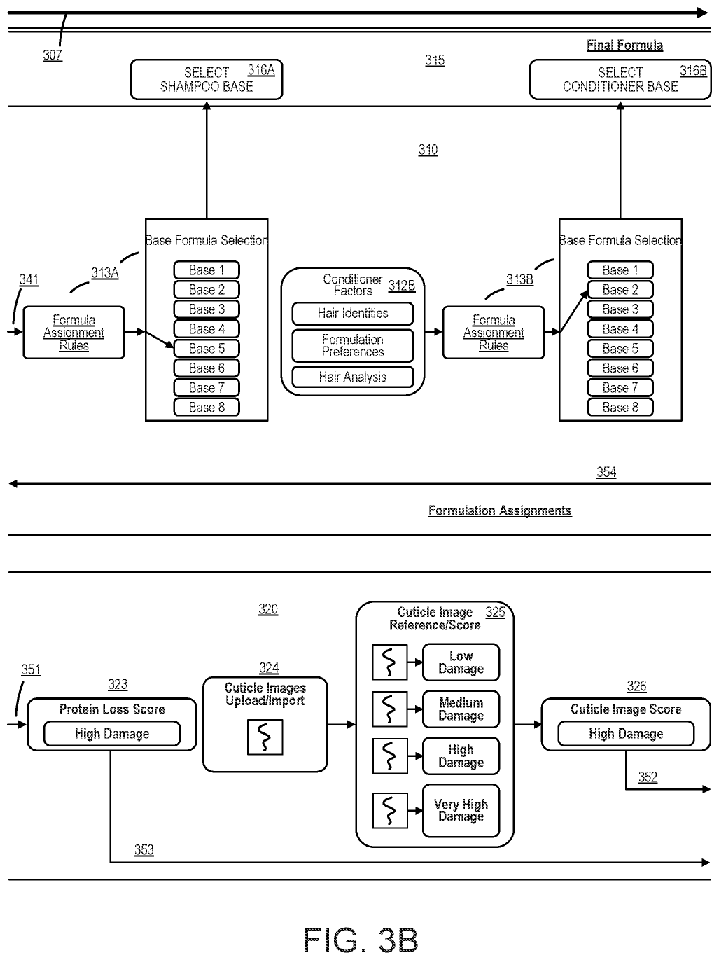

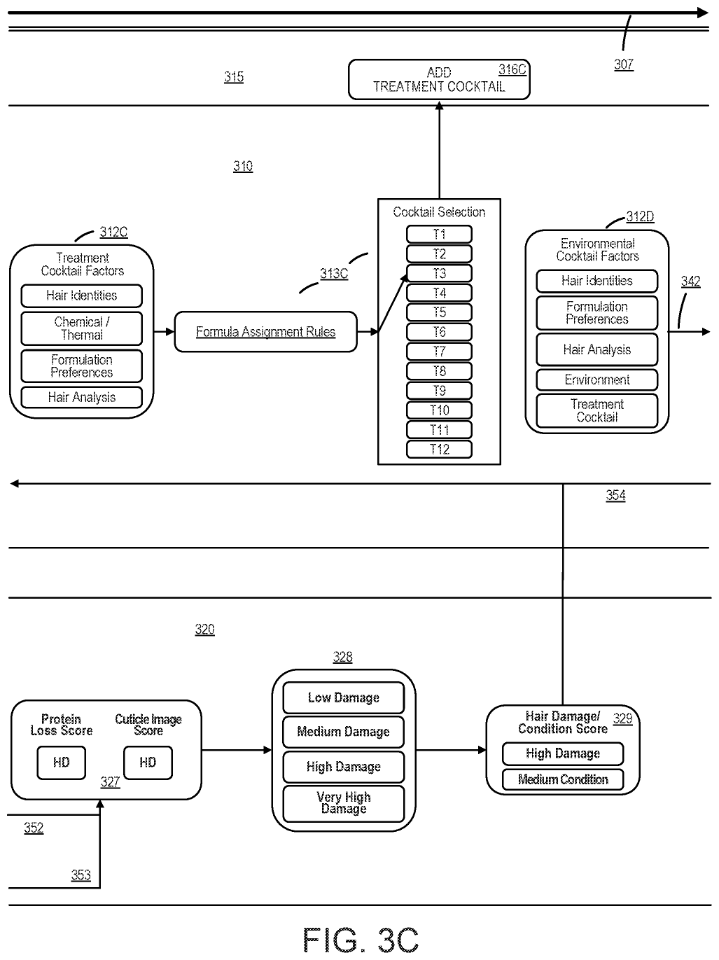

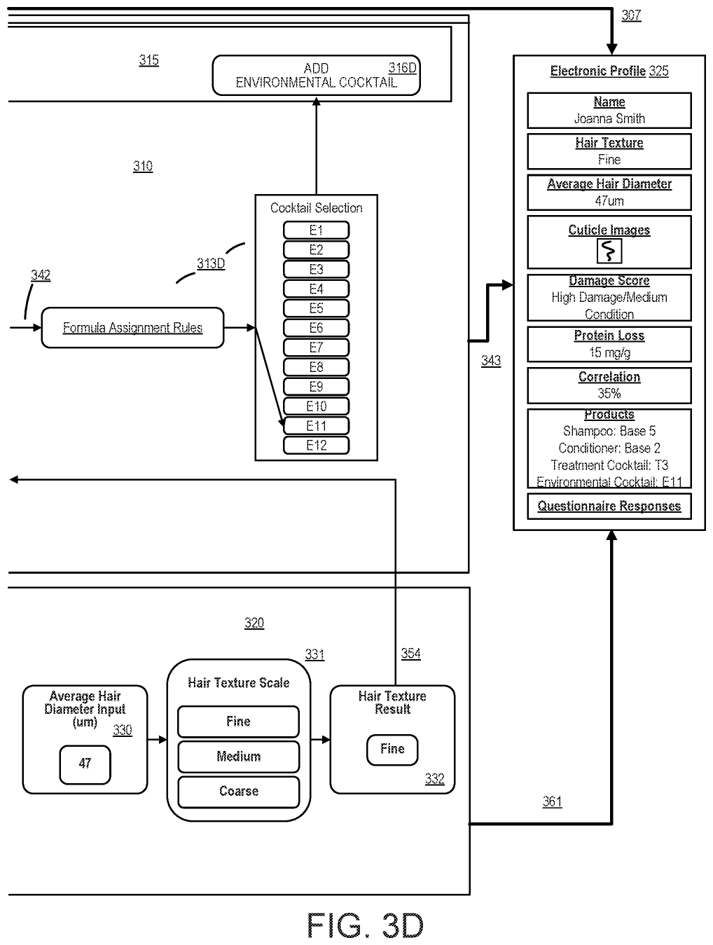

The root cause module 215 maintains data related to root causes, which are factors that affect hair health. The custom hair treatment provider 105 uses user data and hair analysis data to determine values for one or more root cause parameters. The custom hair treatment provider 105 may bucketize root cause parameters into different categories. In an embodiment, there are “Hair Identities,” “Chemical/Thermal,” “Physical,” “Formulation Preferences,” “Hair Analysis,” and “Environment” categories, as illustrated at FIG. 3A (further described below). Hair Identities includes root cause parameters related to physical characteristics of the hair. Chemical/Thermal includes root cause parameters related to past and present treatments performed upon the hair. Physical includes root cause parameters related to hair maintenance. Formulation Preferences includes root cause parameters related to user preferences for the custom hair treatment product. Hair Analysis includes root cause parameters related to results of hair analysis (e.g., hair analysis data). Environment includes root cause parameters related to an environment of the user. The generation of a final formula for a custom hair treatment product by the custom hair treatment provider 105 can involve using different categories of root cause parameters to select different base formulas (e.g., stored formulas for at least part of a custom hair treatment product), as described below.

The treatment formulation module 220 uses root cause parameter values to select one or more base formulas that are components of the final formula for the custom hair treatment product for the user. For example, for one or more selection processes, the treatment formulation module 220 uses some or all root cause parameters to select one base formula from a set of stored base formulas, which may each be associated with a different set of matching root cause parameter values. The treatment formulation module 220 may use, for example, a decision matrix or a machine learning model to select a base formula at each selection process. After performing each selection process, the treatment formulation module 220 combines the selected base formulas to generate a final formula for the custom hair treatment product.

As a particular example, the treatment formulation module 220 may select, using root cause parameter values for a first group of root cause parameter categories, an anti-frizz shampoo base formula; using root cause parameter values for a second group of root cause parameter categories, a high humidity cocktail base formula; and using root cause parameter values for a third group of root cause parameter categories, a protein repair cocktail. The treatment formulation module 220 may make each selection and then combine the various base formulas to produce the custom hair treatment product's final formula, which is a complete formula for the custom hair treatment product.

The machine learning module 225 generates, trains, maintains, and updates machine learning models for the custom hair treatment provider 105. A machine learning model can be any of a variety of machine-learning models, such as a neural network (e.g., a convolutional neural network). The machine learning module 225 may update one or more machine learning models based on user feedback. For example, responsive to user feedback data indicating a satisfaction level with a particular final formula including a particular set of base formulas, the machine learning module 225 may re-train one or more machine learning modules for selection of the particular set of base formulas using the user's root cause parameter values and the user feedback data (e.g., positive feedback increases the weight of the respective root cause parameter values for a particular base formula, and negative feedback decreases the weight of the respective root cause parameter values for the particular base formula).

In an embodiment, the custom hair treatment provider 105 generates a machine learning model to select a base formula in a selection process based on root cause parameter values. The machine learning model takes as input one or more root cause parameter values and outputs scores for each of the base formulas in set of base formulas for the selection process. The machine learning model may be trained on training data comprising sets of root cause parameter values labeled with one or more base formulas, which may each be weighted according to its applicability to the respective set of root cause parameter values (e.g., a most applicable base formula has a greater weight than a base formula that is less applicable).

In an embodiment, the custom hair treatment provider 105 generates a machine learning model to perform imaging analysis upon a hair sample to generate hair analysis data. The custom hair treatment provider 105 either receives an image of user hair or generates an image of user hair (e.g., by applying an imaging sensor to the hair sample). The machine learning model takes as input the image of the user's hair, and outputs one or more values corresponding to one or more root cause parameters (e.g., a cuticle image score). In an embodiment, the input image is a plurality of images of the user hair. The machine learning model may be trained on training data comprising sets of images of hair labeled with one or more values (e.g., cuticle image scores).

The data store 230 stores data of the custom hair treatment provider 105, such as user data, hair analysis data, root cause parameter values, machine learning models and respective training data, user profiles, sets of base formulas, and so on. The data store 230 may be a relational or non-relational database, and may reside at a single computing device, or be distributed across a network of computing devices (e.g., a cloud computing platform).

FIG. 3 is an index figure illustrating the proper arrangement of FIG. 3A-D, which together illustrate a data flow diagram of a technique performed in the system environment, according to an embodiment. As illustrated in FIG. 3, FIG. 3A-D are properly arranged in alphabetical order, from left to right, with each individual sheet adjacent to the next along a long edge of the sheet. Bounding boxes divided across multiple sheets may not precisely align when the respective sheets are placed adjacent to one another. Such changes are made to fit the line drawings to the sheets. Edges of blocks in the figures labeled with similar reference characters and divided across multiple sheets should be interpreted together as if the matching edges on different sheets align.

FIG. 3A-D illustrate a data flow diagram of a technique performed in the system environment 100 by the custom hair treatment provider 105, according to an embodiment. The figures are described together herein as if they were one image, organized as indicated above, and together illustrate the data flow for one embodiment of a technique to generate a custom hair treatment product's final formula. Block 305 indicates user data (e.g., in response to a questionnaire), block 310 indicates formulation assignments, block 315 indicates final formula formulation, and block 320 indicates hair analysis.

In FIG. 3A, the custom hair treatment provider 105 receives user data 305 to the custom hair treatment provider 105 in response to a questionnaire, including an email address, a sample ID, a password, a name, an age, a hair type, a hair length, a natural hair color, hair perception, dryness, haircut frequency, washing frequency, heat styling, products used, color treatments, other treatments, zip code (which may be used to query a third party for environmental data), chlorine exposure, salt water exposure, sun exposure, formulation preferences, scalp sebum level, flaking/irritation, and scent. The user data 305 is used 307 as part of the generation of an electronic profile 325, and is also categorized 306A-F into root cause parameter categories 311A-F. Hair Analysis category 311E also includes hair analysis data 354. Various root cause parameter categories 311 are used for different selection processes. For example, for a selection process for shampoo, shampoo factors 312A include Hair Identities, Chemical/Thermal, Physical, Formulation Preferences, and Hair Analysis root cause parameters. The shampoo factors 312A are evaluated 341 using formula assignment rules to select 313A a base formula for the shampoo 316A. Hair analysis leads to hair analysis data 321, which is used to determine a level of damage 322 to the hair. This determined level 351 is used to determine a protein loss score 323.

In FIG. 3B, the protein loss score 323 is fed into the determination 327 of general set of hair analysis data 327 including a protein loss score and a cuticle image score. An image of the hair is imported 324 and analyzed 325 to determine a cuticle image reference or score 326, which is fed 352 into the general set of hair analysis data. Conditioner factors 312B, a set of root cause parameters, are evaluated using formula assignment rules to select 313B a base formula for the conditioner 316B.

In FIG. 3C, the general set of hair analysis data 327 is evaluated 328 to generate root cause parameter values, such as hair damage and condition scores 329, which form part of the hair analysis data 354. Treatment cocktail factors 312C, a set of root cause parameters, are evaluated using formula assignment rules to select 313C a base formula for a treatment cocktail 316C. Environmental cocktail factors 312D, a set of root cause parameters, are evaluated 342 using formula assignment rules to select 313D an environmental cocktail 316D.

In FIG. 3D, the custom hair treatment provider 105 determines an average hair diameter input 330 as part of hair analysis and determines 331 a hair texture, which is used to determine a hair texture result 332 that forms part of the hair analysis data 354. The hair analysis data is also represented 361 in the electronic profile 325. The final formula 315 including the selected base formulas for shampoo, conditioner, treatment cocktail, and environmental cocktail, is added 343 to the electronic profile 325 as well. The final formula 315 (which may be one formula for a custom hair treatment product that is both a shampoo and conditioner, or may be a plurality of formulas for a custom hair treatment product that includes multiple components, such as a shampoo including a treatment cocktail and a conditioner including an environmental cocktail) may be used to generate a custom hair treatment product for the user.

### Process

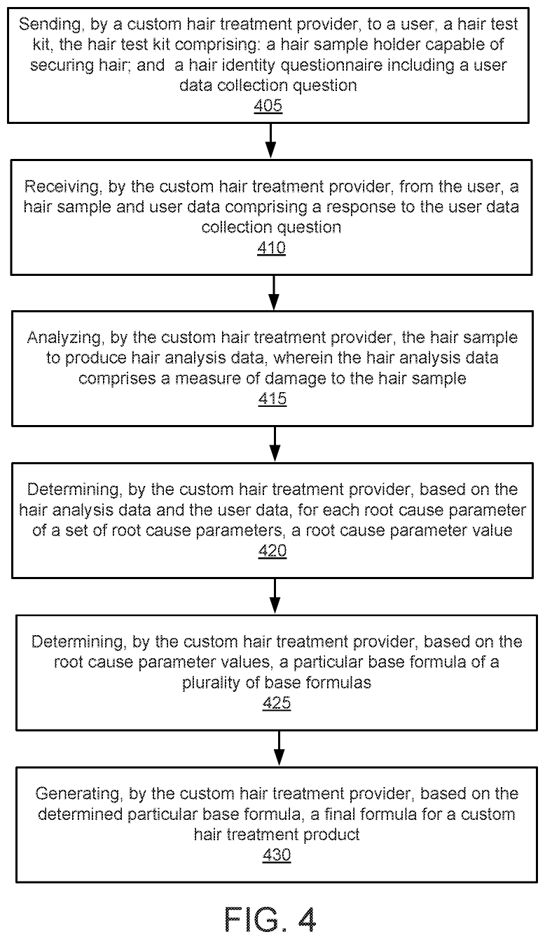

FIG. 4 is a flowchart illustrating steps of a technique performed in the system environment, according to an embodiment. A custom hair treatment provider 105 sends 405, to a user, a hair test kit, the hair test kit comprising a hair sample holder capable of securing hair and a hair identity questionnaire including a user data collection question. The custom hair treatment provider receives 410, from the user, a hair sample and user data comprising a response to the user data collection question. The custom hair treatment provider analyzes 415 the hair sample to produce hair analysis data. The hair analysis data comprises a measure of damage to the hair sample. The custom hair treatment provider determines 420, based on the hair analysis data and the user data, for each root cause parameter of a set of root cause parameters, a root cause parameter value. The custom hair treatment provider determines 425, based on the root cause parameter values, a particular base formula of a plurality of base formulas. The custom hair treatment provider generates 430, based on the determined particular base formula, a final formula for a custom hair treatment product.

In an embodiment, the custom hair treatment provider generates, based on the final formula for the custom hair treatment product, a unit of the custom hair treatment product, and sends, to the user, the unit of the custom hair treatment product.

In an embodiment, the custom hair treatment provider receives, from the user, user feedback data comprising an indicator of user satisfaction with the custom hair treatment product; and adjusts, based on the user feedback data, the final formula for the custom hair treatment product.

In an embodiment, the custom hair treatment provider generates, based on the user data, the hair analysis data, and the final formula for the custom hair treatment product, a user electronic profile. The custom hair treatment provider stores the user electronic profile. The custom hair treatment provider generates, based on the user electronic profile, a user hair report. The custom hair treatment provider sends, to the user, the user hair report.

In an embodiment, the root cause parameters in the set of root cause parameters are divided into a plurality of root cause categories. The custom hair treatment provider determines, based on a first subset of root cause parameter values corresponding to a first subset of root cause categories, the particular base formula for the custom hair treatment product, and determines, based on a second subset of root cause parameter values corresponding to a second subset of root cause categories and comprising a root cause parameter value not in the first subset of root cause parameter values, a second base formula for a second custom hair treatment product for the user.

In an embodiment, determining, based on the set of root cause parameters, the particular base formula of the plurality of base formulas, comprises the custom hair treatment provider applying the root cause parameter values of the set of root cause parameters to a decision matrix, wherein one or more root cause parameters of the set of root cause parameters are criteria of the decision matrix, and one or more base formulas of the plurality of base formulas are alternatives of the decision matrix, and the custom hair treatment provider selecting, as the particular base formula, a base formula of the one or more base formulas corresponding to a highest score in the decision matrix.

In an embodiment, the custom hair treatment provider updates, based on user feedback data, one or more weights of the decision matrix.

In an embodiment, determining, based on the set of root cause parameters, the particular base formula of the plurality of base formulas, comprises the custom hair treatment provider applying the root cause parameter values of the set of root cause parameters to a trained machine learning model, wherein the trained machine learning model generates a score for each base formula of the plurality of base formulas, and the custom hair treatment provider selecting, as the particular base formula, a base formula of the plurality of base formulas with a highest score.

In an embodiment, the trained machine learning model is trained on the plurality of base formulas and respective pluralities of training sets of root cause parameter values, each training set of root cause parameter values having a respective label indicating a level of user satisfaction.

In an embodiment, the custom hair treatment provider receives user feedback data in response to the custom hair treatment product and re-trains, using the user feedback data, the particular base formula, and the root cause parameter values, the trained machine learning model.

In an embodiment, analyzing the hair sample to produce hair analysis data comprises the custom hair treatment provider performing chemical or physical analysis upon the hair sample to produce a total protein loss parameter value for a total protein loss parameter, wherein the measure of damage to the hair sample comprises the total protein loss parameter, where a particular root cause parameter of the set of root cause parameters is based on the total protein loss parameter, and a particular root cause parameter value for the particular root cause parameter is based on the total protein loss parameter value.

In an embodiment, performing the chemical analysis upon the hair sample to produce the total protein loss parameter value comprises assessing protein loss in the hair sample using a protein assay.

In an embodiment, the protein assay comprises extracting surface cuticles from the hair sample, and assessing the protein content of the extract.

In an embodiment, performing the chemical analysis upon the hair sample comprises spectroscopic analysis of the hair sample.

In an embodiment, performing the chemical analysis upon the hair sample comprises optical microscopy on the hair sample.

In an embodiment, performing the chemical analysis upon the hair sample comprises assessing one or more physical properties of hair from the hair sample.

In an embodiment, the one or more physical properties are selected from tensile strength, hair thickness, and hair gloss.

In an embodiment, analyzing the hair sample to produce hair analysis data comprises the custom hair treatment provider performing imaging analysis upon the hair sample to produce a cuticle image parameter value for a cuticle image parameter, wherein the measure of damage to the hair sample comprises the cuticle image parameter, where a particular root cause parameter of the set of root cause parameters is based on the cuticle image parameter, and a particular root cause parameter value for the particular root cause parameter is based on the cuticle image parameter value.

In an embodiment, performing the imaging analysis upon the hair sample to produce the cuticle image parameter value comprises the custom hair treatment provider generating, based on the hair sample, a cuticle image, applying the cuticle image to a trained machine learning model, wherein the trained machine learning model generates a cuticle image score, and determining the cuticle image parameter value based on the cuticle image score.

In an embodiment, the root cause parameters in the set of root cause parameters are divided into a plurality of root cause categories, and the custom hair treatment provider determines, based on a first subset of root cause parameter values corresponding to a first subset of root cause categories, the particular base formula for the custom hair treatment product, and determines, based on a second subset of root cause parameter values corresponding to a second subset of root cause categories and comprising a root cause parameter value not in the first subset of root cause parameter values, a second base formula for the custom hair treatment product, where generating the final formula for the custom hair treatment product is further based on the second base formula.

### DETAILS OF VARIOUS FURTHER EMBODIMENTS

The technology described herein involves a high throughput hair analysis methodology that yields accurate global data from a customer hair sample. Customers simply collect a hair sample from their comb, recent haircut, or in shower shedding, and ship for analysis. The hair is analyzed for cuticle protein changes which provides more dynamic information about the hair quality and integrity.

In an embodiment, the invention may employ measuring cuticle protein changes of a plurality of sample through a standard methodology used in research labs and is directly correlated to the perceived hair properties such as shine, frizz, breakage, and split ends. The benefit of this approach is that it measures that it accounts for cuticle weakening. By using an extraction step, a hair sample's surface layer cuticles are liberated and then the quantity of loss from the sample is correlated.

The invention may be employed for damaged hair samples as well. When hair is damaged, typically there are different degrees of cuticle weakening, meaning two different fibers with different degrees of damage could appear to have the same cuticle condition using standard visual techniques. However, when analyzed for cuticle loss, cuticle integrity may be different relative to the types of conditions the hair sample has been exposed to prior. In addition to analyzing cuticle integrity, the hair that has been exposed to damaging conditions can also experience changes down to the molecular level, and these changes can also be analyzed using chemical and/or physical analysis methods.

In an embodiment, unlike more handheld or in person devices, the technology described in this herein is not limited by the location of the customer nor requires the customer to be present when analysis is conducted. Moreover, the technique described does not require the hair sample to be neatly preserved or aligned in order to capture proper results unlike some of the handheld devices. Since it is already known that humans shed between 50-200 strands of hair daily, the sample process is reduced to simple collection from a brush/comb, a shower drain, and/or freshly cut hair.

In an embodiment, a system is provided that employs a framework for pulling all important data pieces into a single system, the system generating customized hair regiment, products and other recommendations. As a result, an optimized and/or personalized hair care product for a client may be provided. The system may include: a survey means for allowing a customer to complete a survey pertaining to hair of interest; a collection means for collecting a hair sample and/or a scale surface chemical sample for analysis; and an electronic means for identifying hair properties and/or conditions so as to generate an electronic hair profile. The means for identifying hair conditions is typically effective to analyze and quantify hair and scalpproperties and/or conditions. Optionally, a computerized means is provided for moving the electronic hair profile into a software system. In addition, a means may be provided for producing and/or manufacturing hair care products based on the electronic hair profile.

In an embodiment, the invention provides a method for providing a customer with an optimized and/or personalized hair care product. The method may involve: soliciting a customer to complete a survey pertaining to hair of interest; collecting a sample of hair and/or or a sample of scalp chemicals for chemical analysis; electronically identifying the sample of hair's condition and/or property so as to generate an electronic hair profile; moving the electronic hair profile into a software system; and producing hair care products based on the electronic hair profile.

In an embodiment, the invention relates to a kit for providing a consumer with an optimized and/or personalized hair care product. The kit typically includes: a collection means for collecting a hair specimen and/or a surface chemical specimen for analysis, and shipping means for shipping the sample(s) to a location for analyzing the specimen. At least one of the following steps (a)-(c) may be carried out relative to the specimen, e.g., at the location for analyzing the specimen: (a) identifying hair condition and/or properties so as to generate an electronic hair profile; (b) moving the electronic hair profile into a software system; and (c) producing hair care products based on the electronic hair profile. The kit may be used with an online system or a physical retail system.

In an embodiment, a high-throughput system for analyzing a plurality of hair samples, comprising: a plurality of sample holders, each for holding a different hair sample; an analysis means for rapidly effecting analysis of hair held by the sample holders, in series or in parallel; and an electronic means for identifying hair properties so as to generate an electronic hair profile for each sample.

In an embodiment, the high-throughput system further comprises: a computerized means for moving the electronic hair profile into a software system; and a means for producing and/or manufacturing hair products and/or for providing hair recommendations based on the electronic hair profile. In an embodiment, the high-throughput system is effective to provide a plurality of clients/customers with individualized hair products In an embodiment, the analysis means is effective to improve ingredient assignment and/or selection associated with individualized hair products. In an embodiment, the high-throughput system is effective to provide a plurality of clients/customers with personalized hair products and/or services.

In an embodiment, the analysis means is effective to test hair samples for a plurality of types of analysis. In an embodiment, the analysis means is effective to test for mechanical properties of hair samples. In an embodiment, the analysis means is effective to carryout optical microscopy. In an embodiment, the analysis means is effective to carry out chemical analysis. In an embodiment, the analysis means is effective to generate a rating according to a ratings scale to provide customers with a classification for their hair condition.

In an embodiment, a method for providing a plurality of customers with individualized formulated hair products comprises: collecting a sample of hair from each customer; effecting high-throughput analysis of the collected samples of hair; electronically identifying the samples' conditions so as to generate an electronic hair profile for each customer; and formulating different hair care products based on the electronic hair profiles.

In an embodiment, the method further comprises providing the customers with the individualized hair products. In an embodiment, the hair care products are optimized for each customer. In an embodiment, the hair care products are personalized for each hair sample. In an embodiment, the high-throughput analysis effected comprises a plurality of types of hair analysis.

In an embodiment, a method for providing a plurality of consumers with individualized hair care recommendations and/or products associated with each consumer's hair analysis comprises: collecting hair samples from a plurality of consumers; effecting high-throughput analysis of the samples so as to identify a hair condition and/or property for each sample so as to generate an electronic hair profile for each consumer; and providing hair products and recommendations based on the electronic hair profile. In an embodiment, the method further comprises a step of manufacturing the hair products in small batches. In an embodiment, the method further comprises a step of manufacturing the hair products in bulk and sending an appropriate portion of hair products to each consumer with similar electronic hair profiles. In an embodiment, the high-throughput analysis effecting step identifies a plurality of hair conditions and/or properties for each sample so as to generate an electronic hair profile for each consumer.

### Computer Diagram

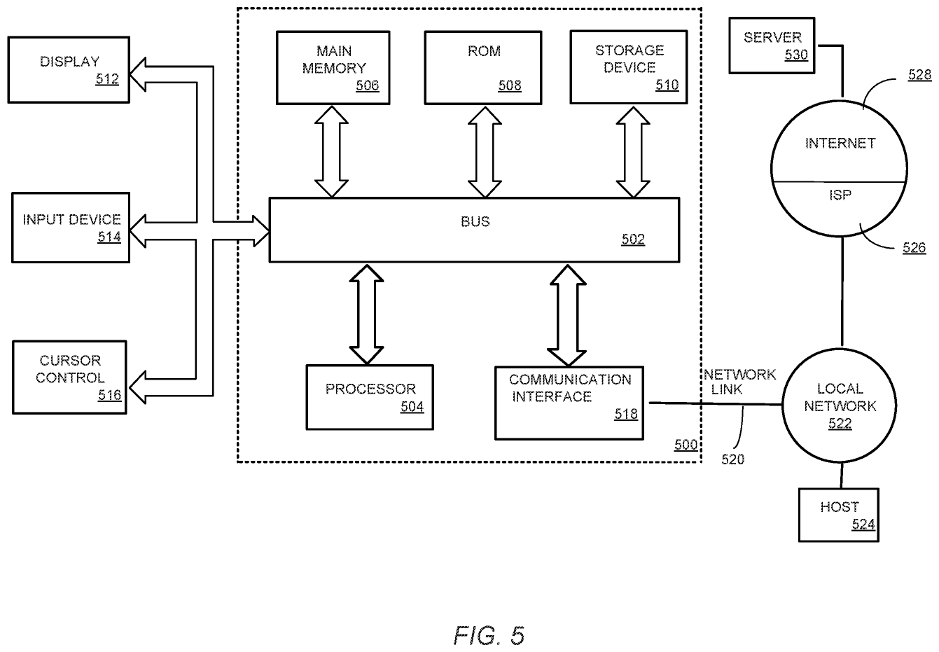

FIG. 5 is a block diagram that illustrates a computer system 500 upon which embodiments of components of the system environment 100 may be implemented. Computer system 500 includes a bus 502 or other communication mechanism for communicating information, and a hardware processor 504 coupled with bus 502 for processing information. Hardware processor 504 may be, for example, a general purpose microprocessor.

Example computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a LCD screen, LED screen, or touch screen, for displaying information to a computer user. An input device 514, which may include alphanumeric and other keys, buttons, a mouse, a touchscreen, or other input elements is coupled to bus 502 for communicating information and command selections to processor 504. In some embodiments, the computer system 500 may also include a cursor control 516, such as a mouse, a trackball, or cursor direction keys for communicating direction information and command selections to processor 504 and for controlling cursor movement on display 512. The cursor control 516 typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and instructions that cause a machine to operation in a specific fashion. Such storage media may comprise non-volatile media and volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic, radio, or light waves, such as those generated during radio-wave and infra-red data communications, such as WI-FI, 3G, 4G, BLUETOOTH, or wireless communications following any other wireless networking standard.

Various forms of media may be involved in carrying one or more sequences of one or more instructions to processor 504 for execution. For example, the instructions may initially be carried on a magnetic disk or solid state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518. The received code may be executed by processor 504 as it is received, and stored in storage device 510, or other non-volatile storage for later execution.

### ADDITIONAL CONSIDERATIONS

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

As used herein, any reference to “one embodiment” or “an embodiment” means that a particular element, label, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the element or component is present unless it is obvious that it is meant otherwise.

Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs that may be used to employ the described techniques and approaches. Thus, while particular embodiments and applications have been illustrated and described, it is to be understood that the described subject matter is not limited to the precise construction and components disclosed. The scope of protection should be limited only by the following claims.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method for generating a custom hair treatment product for a user, the method comprising:
sending, by a custom hair treatment provider, to a user, a hair test kit, the hair test kit comprising:
a hair sample holder capable of securing hair; and
a hair identity questionnaire including a user data collection question;
receiving, by the custom hair treatment provider, from the user, a hair sample and user data comprising a response to the user data collection question;
analyzing, by the custom hair treatment provider, the hair sample to produce hair analysis data, wherein the hair analysis data comprises a measure of damage to the hair sample;
determining, by the custom hair treatment provider, based on the hair analysis data and the user data, for each root cause parameter of a set of root cause parameters, a root cause parameter value;
determining, by the custom hair treatment provider, based on the root cause parameter values, a particular base formula of a plurality of base formulas; and
generating, by the custom hair treatment provider, based on the determined particular base formula, a final formula for a custom hair treatment product.

2. The method of claim 1, further comprising:
generating, by the custom hair treatment provider, based on the final formula for the custom hair treatment product, a unit of the custom hair treatment product; and
sending, by the custom hair treatment provider, to the user, the unit of the custom hair treatment product.

3. The method of claim 2, further comprising:
receiving, by the custom hair treatment provider, from the user, user feedback data comprising an indicator of user satisfaction with the custom hair treatment product; and
adjusting, by the custom hair treatment provider, based on the user feedback data, the final formula for the custom hair treatment product.

4. The method of claim 1, further comprising:
generating, by the custom hair treatment provider, based on the user data, the hair analysis data, and the final formula for the custom hair treatment product, a user electronic profile;
storing, by the custom hair treatment provider, the user electronic profile;
generating, by the custom hair treatment provider, based on the user electronic profile, a user hair report; and
sending, by the custom hair treatment provider, to the user, the user hair report.

5. The method of claim 1, wherein the root cause parameters in the set of root cause parameters are divided into a plurality of root cause categories, the method further comprising:
determining, by the custom hair treatment provider, based on a first subset of root cause parameter values corresponding to a first subset of root cause categories, the particular base formula for the custom hair treatment product; and
determining, by the custom hair treatment provider, based on a second subset of root cause parameter values corresponding to a second subset of root cause categories and comprising a root cause parameter value not in the first subset of root cause parameter values, a second base formula for a second custom hair treatment product for the user.

6. The method of claim 1, wherein determining, by the custom hair treatment provider, based on the set of root cause parameters, the particular base formula of the plurality of base formulas, comprises:
applying, by the custom hair treatment provider, the root cause parameter values of the set of root cause parameters to a decision matrix, wherein one or more root cause parameters of the set of root cause parameters are criteria of the decision matrix, and one or more base formulas of the plurality of base formulas are alternatives of the decision matrix; and
selecting, by the custom hair treatment provider, as the particular base formula, a base formula of the one or more base formulas corresponding to a highest score in the decision matrix.

7. The method of claim 6, further comprising:
updating, by the custom hair treatment provider, based on user feedback data, one or more weights of the decision matrix.

8. The method of claim 1, wherein determining, by the custom hair treatment provider, based on the set of root cause parameters, the particular base formula of the plurality of base formulas, comprises:
applying, by the custom hair treatment provider, the root cause parameter values of the set of root cause parameters to a trained machine learning model, wherein the trained machine learning model generates a score for each base formula of the plurality of base formulas; and
selecting, by the custom hair treatment provider, as the particular base formula, a base formula of the plurality of base formulas with a highest score.

9. The method of claim 8, wherein the trained machine learning model is trained on the plurality of base formulas and respective pluralities of training sets of root cause parameter values, each training set of root cause parameter values having a respective label indicating a level of user satisfaction.

10. The method of claim 8, further comprising:
receiving, by the custom hair treatment provider, user feedback data in response to the custom hair treatment product; and
re-training, by the custom hair treatment provider, using the user feedback data, the particular base formula, and the root cause parameter values, the trained machine learning model.

11. The method of claim 1, wherein analyzing, by the custom hair treatment provider, the hair sample to produce hair analysis data, comprises:
performing, by the custom hair treatment provider, chemical or physical analysis upon the hair sample to produce a total protein loss parameter value for a total protein loss parameter, wherein the measure of damage to the hair sample comprises the total protein loss parameter;
wherein a particular root cause parameter of the set of root cause parameters is based on the total protein loss parameter, and a particular root cause parameter value for the particular root cause parameter is based on the total protein loss parameter value.

12. The method of claim 11, wherein performing the chemical analysis upon the hair sample to produce the total protein loss parameter value comprises assessing protein loss in the hair sample using a protein assay.

13. The method of claim 11, wherein the protein assay comprises extracting surface cuticles from the hair sample, and assessing the protein content of the extract.

14. The method of claim 11, wherein performing the chemical analysis upon the hair sample comprises spectroscopic analysis of the hair sample.

15. The method of claim 11, wherein performing the chemical analysis upon the hair sample comprises optical microscopy on the hair sample.

16. The method of claim 11, wherein performing the chemical analysis upon the hair sample comprises assessing one or more physical properties of hair from the hair sample.

17. The method of claim 16, wherein the one or more physical properties are selected from tensile strength, hair thickness, and hair gloss.

18. The method of claim 1, wherein analyzing, by the custom hair treatment provider, the hair sample to produce hair analysis data, comprises:
performing, by the custom hair treatment provider, imaging analysis upon the hair sample to produce a cuticle image parameter value for a cuticle image parameter, wherein the measure of damage to the hair sample comprises the cuticle image parameter;
wherein a particular root cause parameter of the set of root cause parameters is based on the cuticle image parameter, and a particular root cause parameter value for the particular root cause parameter is based on the cuticle image parameter value.

19. The method of claim 18, wherein performing the imaging analysis upon the hair sample to produce the cuticle image parameter value comprises:
generating, by the custom hair treatment provider, based on the hair sample, a cuticle image;
applying, by the custom hair treatment provider, the cuticle image to a trained machine learning model, wherein the trained machine learning model generates a cuticle image score; and
determining, by the custom hair treatment provider, the cuticle image parameter value based on the cuticle image score.

20. The method of claim 1, wherein the root cause parameters in the set of root cause parameters are divided into a plurality of root cause categories, the method further comprising:
determining, by the custom hair treatment provider, based on a first subset of root cause parameter values corresponding to a first subset of root cause categories, the particular base formula for the custom hair treatment product; and
determining, by the custom hair treatment provider, based on a second subset of root cause parameter values corresponding to a second subset of root cause categories and comprising a root cause parameter value not in the first subset of root cause parameter values, a second base formula for the custom hair treatment product;
wherein generating the final formula for the custom hair treatment product is further based on the second base formula.
